(19) United States
(12) Patent Application Publication
(10) Pub. No.: US 2004/0022857 A1
(43) Pub. Date: Feb. 5, 2004

(54) Synthesis of highly conjugated polymers

(76) Inventors: Egidijus E. Uzgiris, Niskayuna, NY (US); Bahram Moasser, Schenectady, NY (US); Kenneth M. Fish, Clifton Park, NY (US); Joanne F. Smith, Ballston Lake, NY (US)

Correspondence Address: Raymond E. Farrell, Esq., Carter, DeLuca, Farrell & Schmidt, LLP, Suite 225, 445 Broad Hollow Road, Melville, NY, 11747, US

(21) Appl. No.: 10/209,726

(22) Filed: Jul. 31, 2002

(51) Int. Cl. A61K 9/14

(52) U.S. Cl. 424/486

(57) ABSTRACT

Improved methods for forming substituted polymers having a high degree of conjugation include the steps of activating a steric hindrance molecule under conditions that ensure substantially all the steric hindrance molecules are mono-activated and then reacting with a polymer. An improved method for recovering polypeptides substituted with steric hindrance molecules and having an elongate configuration removes impurities and volatiles without achieving complete dryness of the copolymer.

### BACKGROUND

1. Technical Field

The present disclosure relates to improved methods for producing highly conjugated polymers that are useful, for example, for diagnostic imaging and drug delivery.

2. Description of Related Art

The use of chemical contrast agents greatly enhances the utility and scope of magnetic resonance imaging (MRI).

Typical contrast agents contain molecular complexes that include paramagnetic lanthanide ions (such as Gadolinium III) attached to carrier molecules. The high electron spin state of these ions creates an oscillating magnetic field that perturbs the T1 and T2 of the hydrogen atoms of tissue water that is in contact with the ion.

Contrast in a specific tissue (e.g., tumor tissue) is achieved by the higher affinity of those tissues for the molecular complex. The effectiveness of a contrast agent depends on the selective uptake of the carrier molecule in the targeted tissue, the delivery of a high concentration of the paramagnetic ion to the tissue, the long lifetime of the carrier molecule in the targeted tissue, and efficient elimination of the molecule with no toxic consequences.

One known contrast agent contains carrier molecules which are polypeptides having a diameter larger than pores of blood vessels of normal tissue and smaller than pores of blood vessels of tumor tissue. See, U.S. Pat. No. 5,762,909. These carriers have a length several orders of magnitude greater than their diameter, a net negative charge, and form a worm-like chain conformation with a long persistence length. Lanthanide complexes (e.g., gadolinium-diethylenetriamine pentaacetic acid complexes) are attached to these carrier molecules to create complex molecules which are introduced into a blood vessel of the subject.

These complex molecules pass though the pores of only the tumor tissue and interact with the fibrous structures of the tumor interstitium. The penetration of the tumor interstitium by the complex molecules is enhanced by the worm-like configuration of the complex molecule which allows the molecule to “snake” around fixed obstacles in the extracellular matrix of the tumor interstitium by a mechanism of polymer reptation.

The worm-like configuration of the complex molecule is achieved by attaching a sufficient number of diethylenetriamine pentaacetic acid (DTPA) molecules along the polypeptide chain to eliminate or reduce intra-chain charge interactions as well as restrict rotation about a bond at each peptide link. The amount of substitutions (also referred to as the degree of conjugation) thus affects the configuration of the resulting complex, with a higher degree of conjugation providing a more consistent structure and better targeting.

Accordingly, it would be advantageous to provide polypeptide-DTPA molecules that have the highest degree of conjugation possible for use as contrast agents.

### SUMMARY

An improved method for forming conjugated polymeric molecules is provided herein. The present methods provide substituted polymers having a high degree of conjugation. The methods include the steps of activating a steric hindrance molecule under conditions that ensure substantially all the steric hindrance molecules are mono-activated. The mono-activated steric hindrance molecules are then reacted with a polymer, such as, for example, a polypeptide. The resulting polymer-steric hindrance molecule copolymer is then recovered. It has unexpectedly been discovered that the presence of multi-activated steric hindrance molecules in prior art processes increases the presence of inter- or intra-molecularly folded conformations, thereby inhibiting a high degree of conjugation from being consistently achieved.

In another aspect, an improved method for recovering highly conjugated polypeptides is described herein. At the end of the synthesis procedure, the desired conjugated polypeptide is included in a reaction mixture that includes impurities, volatile solvents and water. The present purification method removes impurities and volatiles without achieving complete dryness of the conjugated polypeptide. It has unexpectedly been determined that excessive dryness affects the configuration of the conjugated polypeptide and interferes with the determination of degree of conjugation. The purification method includes the steps of removing one or more volatile components using vacuum, centrifuging the remaining mixture, filtering to separate the conjugated polypeptide-containing filtrate from the liquid fraction containing impurities, and performing dialysis to recover the conjugated polypeptide. Optionally, ultrafiltration of the dialyzed product may be performed.

The highly conjugated polymers described herein are useful as drug delivery agents and as contrast agents for medical imaging, such as, for example, MRI.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawing, in which:

FIG. 1 is a reaction scheme for preparing highly conjugated polymers in accordance with one embodiment of this disclosure.

FIG. 2 is an illustration of inter-strand and intra-strand cross-linking of polypeptides.

FIG. 3 an illustration of a highly substituted polypeptide according to the present invention.

### DETAILED DESCRIPTION OF THE INVENTION

The present methods increase the degree of conjugation of substituted polymers compared to prior art processes. The higher degree of conjugation obtained by the present methods improves the effectiveness of the substituted polymers as carriers for active substituents, such as, for example, contrast agents or drugs.

The first step in the present methods is providing a substantially mono-activated steric hindrance molecule (“SHM”). The term “activated” means that a functional group is provided on the SHM which permits covalent bonding of the molecule to a polymer chain. By the term “substantially mono-activated” it is meant that about 90% or more of the steric hindrance molecules contain only a single activated site.

The SHM is any molecule that by its physical size enforces a elongated conformation by providing steric hindrance between neighboring steric hindrance molecules. Preferably the SHM is neutral in charge or presents negative charges in an aqueous environment along the polymer chain to assist in keeping the polymer backbone straight through Coulombic repulsion.

In particularly useful embodiments, the SHM contains or chelates an imaging producing entity. Suitable imaging producing entities include paramagnetic entities, entities which undergo nuclear reaction resulting in release of detectable radiation. Non-limiting examples include ions which release alpha particles, gamma particles, beta particles, or positrons. Such image producing entities are known to those skilled in the art. Gamma emitters include, for example, ¹¹¹In and ¹⁵³ Gd. Positron emitters include, for example, ⁸⁹ Zr, which may be employed in positron emission tomography (PET) imaging.

Particularly preferred steric hindrance molecules are molecules that chelate with paramagnetic entities. As those skilled in the art will appreciate, paramagnetic entities include certain transition metals and lanthanide ions. Any molecule known to complex with paramagnetic entities and which is of sufficient size to provide steric hindrance against polymer bending can be used as the SHM. Preferably, the SHM has a net negative charge. Suitable lanthanide ion chelating molecules include, but are not limited to diethylenetriaminepentaacetic acid (DTPA), 1,4,7,10-Tetraazacyclododecane-1,4,7,10-tetraacetic acid (DOTA), 1,4,7,10Tetraazacyclododecane-1,4,7,10-tetrakis(2-propionic acid) (DOTMA), 1,4,8,11-Tetraazacyclotetradecane-1,4,8,11-tetraacetic acid (TETA), 1,4,7,10-Tetraazacyclododecane-1,4,7,10-tetrakis[3-(4-carboxyl)-butanoic acid], 1,4,7,10-Tetraazacyclododecane-1,4,7,10-tetrakis(acetic acid-methyl amide), 1,4,7,10-Tetraazacyclododecane-1,4,7,10-tetrakis(methylene phosphonic acid), and p-Isothiocyanatobenzyl-1,4,7,10-tetraazacyclododecane-1,4,7,10-tetraacetic acid (pSCN-Bz-DOTA). Ligands useful for chelating for other ions (such as, for example, Fe(III), Mn(II), Cu(II), etc.) include Bis(thiosemicarbazone) and derivatives, Porphyrins and derivatives, 2,3-Bis(2-thioacetamido) propionates and derivatives, N,N′-bis(mercaptoacetyl)-2,3-diaminopropanoate, and B is (aminoethanethiol) and derivatives.

The activating group present on the SHM can be any group which will react with a polymer. Suitable groups include, but are not limited to mixed carbonate carbonic anhydride groups, succinimidyl groups, amine groups and dicyclohexylcarbodiimide (DCC) groups. Those skilled in the art will readily envision reaction schemes for attaching an activating group to any given SHM.

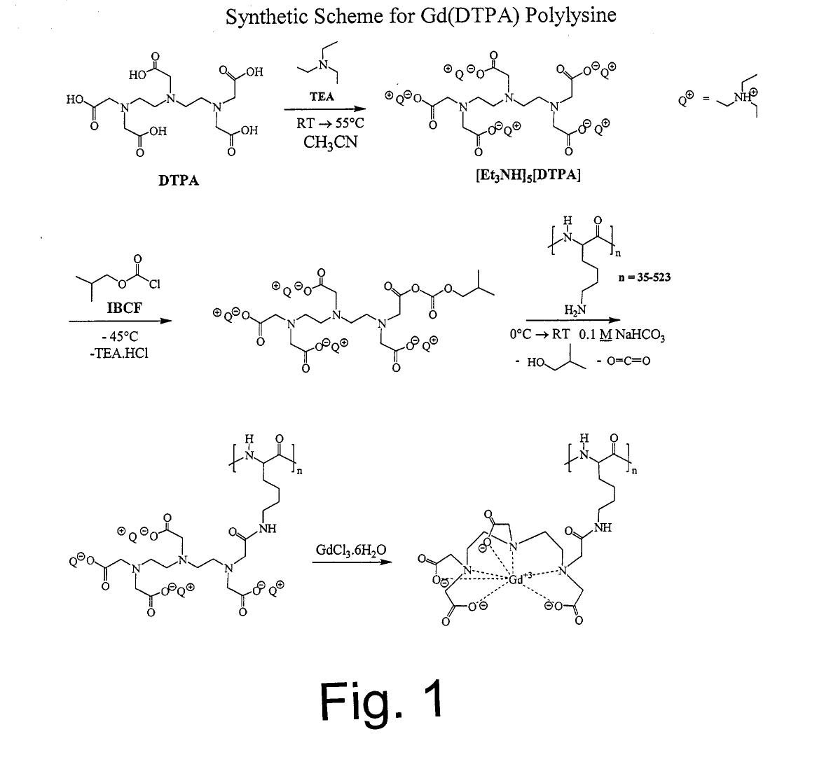

In particularly useful embodiments, the SHM is DTPA and the activating groups are mixed carbonate carbonic anhydride groups. A typical reaction scheme for activationg DTPA and reacting it with a polypeptide backbone is shown in FIG. 1. As seen therein, a monoanhydride-DTPA is first prepared. Specifically, a flask is charged with acetonitrile and DTPA. Triethylamine is then added via syringe. The solution is warmed to 60° C. under a nitrogen atmosphere. The mixture is stirred until homogeneous. The clear solution is then cooled to −45° C. under nitrogen atmosphere and isobutyl chloroformate is slowly added to result in the monoanhydride of DTPA. As those skilled in the art will appreciate, DTPA has five acid groups available for conversion to anhydride. However, since substantially mono-activated DTPA is desired, only one of these acid sites should be converted to anhydride. It has unexpectedly been found that the slow addition of the chloroformate while cooling below −40° C. accomplishes this result, i.e., that about 90% or more of the DTPA is a monoanhydride of DTPA.

The substantially mono-activated SHM is then reacted with a polymer. The nature of the polymer is not critical, provided that the polymer has pendant groups which can be reacted with the activated SHM to provide a polymer-SHM copolymer having an elongated structure. Suitable pendant groups which may be present in the polymer include, but are not limited to amine groups, carboxyl groups and hydroxyl groups. Useful polymers include homo- and co-polymers of poly(amino acids), poly(vinyl amine), poly(4-aminostyrene), poly(acrylic acid), poly(methacrylic acid), poly(carboxynorbornene), and dextran. Preferably, the polymer is a polypeptide. The polypeptide can be an amino acid homopolymer or a copolymer of two or more amino acids. Preferably, the polypeptide is selected from the group consisting of polylysine, polyglutamic acid, polyaspartic acid, copolymers of lysine and either glutamic acid or aspartic acid. Other polymers may be used provided that after reaction with the SHM, the resulting copolymer has an elongated structure characterized by a molecular length that is 5 to 500 times the cross-sectional diameter of the copolymer molecule. In addition, for certain applications the resulting copolymer should also have a net negative charge. Where the product is to be used for medical imaging, the polymer preferably is of sufficient length to increase the time in which the product circulates in the blood. Clearance from the blood is rapid for short molecules, resulting in a short plasma lifetime. Plasma lifetime increases rapidly as the polymers increase in length. For example, where the polymer is a polypeptide, a plateau is reached for a molecular length of about 500 residues and little further change in lifetime occurs.

The precise conditions for reacting the polymer with the substantially mono-activated SHM will depend upon a number of factors including the particular polymer chosen and the specific SHM used. Those skilled in the art will readily envision reaction schemes for any given pair of materials to produce the desired polymer-SHM copolymers.

In a particularly useful embodiment, for example, the monoanhydride-DTPA described above is simply added dropwise to an aqueous solution of polylysine under ambient atmospheric conditions.

In another example, where the reactive pendant groups on the polymer backbone are electrophilic groups (such as, for example, a carboxylic acid groups), the anhydride of DTPA described above can be reacted overnight with a diamine (in which the diamine is in large excess to the anhydride). Ethylene diamine is a suitable choice, giving in the end a DTPA linkage of the desired length to achieve proper steric hindrance against polymer chain bending. The product is separated from the diamine and from DTPA which was not reacted, by ion exchange chromatography. The product is substantially mono-amine DTPA. Where the substantially mono-activated steric hindrance molecule is the foregoing monoamine-DTPA, it can be linked to a carboxyl group contining polymer (such as, for example, poly-glutamic acid) by a carboxyl coupling method. The carboxy acid groups of the polymer are activated by a coupling reagent, such as, for example, 1 Ethyl-3-(3-Dimethylaminopropyl) carbodiimide Hydrochloride (EDC) (Pierce, Rockford, Ill.). The activated carboxy acid groups on the polymer are then combined with the monoamine-DTPA to produce an amide linkage of the DTPA to the polymer backbone as a sidechain which acts as a steric hindrance straightening the polymer backbone.

The resulting polymer-steric hindrance molecule copolymer is then purified. During purification, the polymer-SHM copolymer is separated from the volatile solvents and other impurities. Any known techniques can be used to purify the polymer-SHM copolymers.

In a particularly useful embodiment, where a polypeptide backbone is used, a purification scheme is employed which does not result in complete drying of the polymer-SHM copolymer. It has unexpectedly been determined that excessive dryness affects the configuration of the copolymer and interferes with the determination of degree of conjugation.

A preferred purification scheme involves first exposing the reaction mixture to reduced pressure to remove impurities that are more volatile than water. Care should be taken not to remove all water from the reaction mixture during this step. The next step in this preferred purification scheme is to centrifuge the remaining reaction mixture. Soluble impurities remain in the supernatant fluid. The retentate from the centrifuge step is resuspended and subjected to dialysis. Optionally, ultrafiltration is performed on the dialyzed polymer. Techniques for these processes are within the purview of those skilled in the art.

The resulting product can then be characterized using any technique known to those skilled in the art, such as, for example, high performance liquid chromatography (HPLC).

In certain embodiments where the conjugated polymers are intended to be used as imaging agents, an image producing entity is incorporated into the conjugated polymer. Thus, for example, to achieve a MR active agent, a paramagnetic ion (such as, for example, gadolinium) can be incorporated into the product polymer chelating DTPA groups by dropwise addition of a gadolinium salt, such as, for example gadolinium chloride or gadolinium citrate. The dropwise addition of Gd continues until a slight indication of free Gd (not chelated by available DTPA groups) is noted (small aliquots of polymer solution added to 10 microMolar of arzenzo IIII in acetate buffer—free Gd turns the dye solution blue). The Gd-loaded highly conjugated polymer is then ready for introduction into a blood vessel of the subject.

In certain embodiments, the conjugated polymer can be used for drug delivery. It is contemplated, for example, that the SHM can itself be a therapeutic agent. It is also contemplated that a therapeutic agent can be attached at a few sites along the substituted polymer chain. By way of example, chemotherapeutic agents (such as, for example, doxorubicin) which have been shown to have activity against tumors can be attached to the conjugated polymer. For therapy, one could also use a radiotherapicutic agent such as a beta emitters, an isotope of Yttrium, ⁹⁰ Y, or Astatine, ²¹¹ At, which also emits alpha particles. The therapeutic entity can be attached to the conjugated polymer using techniques known to those skilled in the art. It is also contemplated that therapeutic agents can be used in combination with other types of active agents incorporated into the conjugated polymer. For example, the polymer backbone can be highly conjugated with a non-therapeutic SHM which chelates an imaging agent and a therapeutic agent can appear at only a few sites along the backbone. As another example, the polymer backbone can be highly conjugated with a non-therapeutic SHM, and a therapeutic agent can be bound to the SHM, rather than being bound directly to the polymer backbone.

In certain embodiments, the conjugated polymer can have a targeting moiety attached thereto. It is contemplated, for example, that the SHM can itself be a targeting moiety. It is also contemplated that a targeting moiety can be attached at a few sites along the substituted polymer chain. The targeting moiety can be attached to the conjugated polymer using techniques known to those skilled in the art. It is also contemplated that targeting moieties can be used in combination with other types of active agents incorporated into the conjugated polymer. For example, the polymer backbone can be highly conjugated with a non-therapeutic SHM which chelates an imaging agent and a targeting moiety can appear at only a few sites along the backbone. As another example, the polymer backbone can be highly conjugated with a non-therapeutic SHM, and a targeting moiety can be bound to the SHM, rather than being bound directly to the polymer backbone.

Where the complex molecules formed using the present methods are intended to be used for tumor imaging, the molecules preferably have a cross sectional diameter in the range of about 20 Angstroms (A) to about 50 A. This range provides molecules which are larger than that of the pores of normal tissue such that they are contained within the blood vessels in normal tissue but have a cross sectional diameter smaller than that of the pores of the vessels in tumor tissue such that they may readily pass out of the pores and into the interstitial space. In this manner, products made using the methods herein can be advantageously used for tumor imaging or therapy.

In particularly useful embodiments, the molecules are formed using a polypeptide backbone and the conformation provided by the present methods is that of a worm-like shape being essentially a stretched out, extended chain with little folding. A measure of the “straightness” of a molecule is a persistence length. Preferably the polypeptide is chosen to provide a persistence length in the range of about 100 Å to about 600 Å. Persistence length is related to a radius of gyration, measured by light scattering experiments. A folded polypeptide such as poly-L-lysine (PLL) with little or no substitution, has a low persistence length of about 10 Å, and is not suitable for targeting tumor tissue. Complex molecules with a persistence lengths of 100-200 Å concentrate much more readily in tumor tissue.

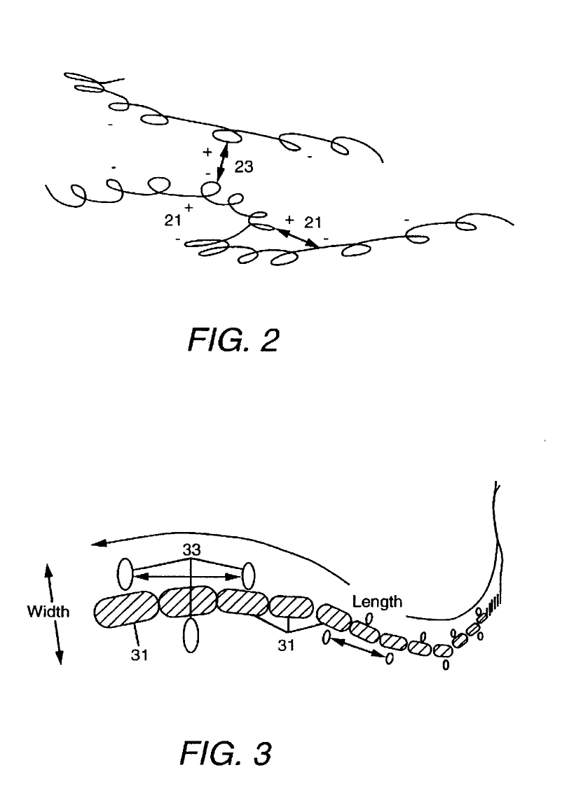

An elongated, worm-like conformation of a macromolecule results in greater uptake than other conformations, such as folded, or globular conformations. Conformation may be measured by a persistence length of the molecule. This may be determined by light scattering. Conformation is a result of intra-chain charge interaction, and rigidity of the molecule. As those skilled in the art will appreciate, many polypeptides tend to fold into tight random coils due to the relatively free rotation around each peptide bond. Also, if each polypeptide is composed of oppositely charged amino acids, then intra-chain charge interaction as shown by bond 21 in FIG. 2. Inter-chain charge interaction between chains may also occur as shown by bond 23 of FIG. 2. If there is significant intra-chain charge interactions, the complex molecules may assume a globular, or folded, conformation.

It is sometimes difficult to measure the persistence length of certain molecules by light scattering to determine their conformation because of the effects of contaminant particles in the test solutions. However, by measuring the magnetic resonance (MR) T₁ relaxation of a paramagnetic entity attached to the carrier, one could determine the conformation of the molecules of interest. This is performed by attaching a magnetic resonance (MR) active entity, such as gadolinium, to a carrier molecule extending as a sidechain. Usually this is performed by encapsulating the MR active entity in a lanthanide ion-complexing molecule. When the carrier molecule is in an elongated conformation, the chelator/MR active entity is free to rotate about its attachment point to the main chain, allowing a long T₁ relaxation time of the surrounding water protons which are the source of the MR signal. When the carrier molecule is in a globular or highly folded conformation, steric hindrance, and molecular crowding causes interaction with the chelator/MR active entity restricting rotation about its bond to the main chain. Thus, the chelator/MR active entity moves only with the general slow motion of the carrier molecule. This produces a short T₁ relaxation time.

Therefore, a high relaxivity is associated with a molecule which folds upon itself into a globular conformation, such as albumen, at about 15 sec.⁻³ milliMolar⁻¹ (sec.⁻¹ mM⁻¹). A low relaxivity is associated with an elongated molecule such as highly substituted Gd-DTPA PLL^{h} in which the Gd can rotate rapidly, having a relaxivity of about 8 sec.⁻¹ mM⁻¹. The optimum conformation of the present invention is associated with a relaxivity of 7-8 sec.⁻¹ mM⁻¹. When the relaxivity of a peptide agent is high, the uptake coefficient of such an agent is invariably low, evidently due to the absence of the reptation mechanism.

Since many in-vivo chemical entities have a negative charge, molecules introduced into the subject must have a net negative charge to reduce agglutination and to allow for stable long circulation in the blood plasma.

In FIG. 3 a polypeptide carrier having a plurality of side chains substituting the hydrogen atoms is shown. The polypeptide is comprised of a plurality of amino acids 31, each linked end to end through a polypeptide bond. A plurality of side residues 33 are attached which cause steric hindrances and repulsion to straighten the polypeptide chain.

FIG. 3 also shows that the length of the polypeptide should be significantly longer than its diameter by approximately 2 orders of magnitude. This causes the polypeptide and any attached chemical entities to pass through pores in tumor tissue and become trapped the tumor interstitium as discussed above.

### EXAMPLE

Gd-DTPA-Synthesis

Under an inert atmosphere, the penta anion of DTPA was prepared by reaction of DTPA (2.97 g, 7.56 mmol) with triethylamine (5.37 ml, 3.9 g, 38.56 mmol) in 35 ml acetonitrile for 50 min. and 55° C. Isobutylchloroformate (1.10 ml, 1.16 g, 8.47 mmol) was added dropwise to the DTPA penta anion, cooled in an well-equilibrated −45° C. bath, maintained by a Cryotrol temperature controller (Thermo NESLAB, Portsmouth, N.H.). After stirring at this temperature for 1 hour, the resulting thick slurry of the diethylenetriamine tetraaceticacid-isobutyl dianhydride was added dropwise, under ambient atmospheric conditions, to 15 ml of an aqueous 0.1 M NaHCO₃ buffered pH 9 solution of poly-L-lysine (DP=402, MW=84,000 gmol⁻¹, Mw/Mn=1.10, 0.25 g, 1.2 mmol lysine residue) at 0° C.

After 16 hours of stirring at ambient temperature most (if not all) of the acetonitrile was removed under high vacuum (˜10 micronsHg) over a 20-25 min period. A warm water bath was used to maintain uniform temperature, prevent sample bumping and inhibit vacuum cooling. The resulting solution was centrifuged twice at 5000 rpm and 5° C. to deposit a thick semi-translucent sediment. The supernatant containing the product was purified by dialysis and sometimes further purified by ultrafiltration. The resulting DTPA-polylysine was labeled using hydrated Gd(citrate) at lower pH. The efficacy of conjugation was determined by a colorimetric test for the identification of underivatized polylysine amine. Polymer purity was determined by HPLC. Typical values for conjugation ranged from 92-98%. Typical polymer yields raged from 40-60%.

All glassware used in the preparation of the dianhydride was dried by heating under a nitrogen atmosphere. Acetonitrile was distilled from calcium hydride and stored over 4-angstrom molecular sieves. High purity triethylamine and isobutylchloroformate were employed and were stored under inert atmosphere. The dianhydride was prepared in a Morton flask using a mechanical overhead stirrer for achieving high mixing efficiency. Finally, the synthesis up to the DTPA polylysine conjugate was carried out uninterrupted. If necessary, the final polymer can be indefinitely stored at 4° C.

Gd-DTPA-Polylysine Purification

Dialysis:

The polymer solution was loaded into 5 mL regenerated cellulose disposable dialyzers with a molecular weight cut off of 8000(Sigma-Aldrich cat# Z36,849-0). The polymer solutions were dialyzed using a Spectra/Por EZ-1 Multidialyzer, against approximately 2 liters of 10 mM NaHCO3 for 24 hours, with constant motion. The buffer was changed after 4-6 hours. The samples were dialyzed for 24 hours. Initial and final dialyzed volumes were noted. The initial and final dialyzed polymer solutions were analyzed by HPLC, without filtering.

Ultrafiltration:

The following devices were used for these experiments: Amicon Centriplus YM-3 centrifugal filter devices, containing a regenerated cellulose membrane with a molecular weight cut off of 3000(cat # 4420). The membranes were pre-washed with 50 mM phosphate buffer, pH 7 before use to remove polyethylene glycol. The washing procedure was as follows: Add 14 mL of phosphate buffer to the top of the device. Spin for one hour at 3500 RPM in a Sorvall RC-5B Refrigerated Superspeed Centrifuge refrigerated centrifuge at 110° C. (, Dupont Corp., Wilmington Del.). Phosphate buffer from top and bottom of the device was replenished after the washing step. Fresh buffer was added and centrifuged as before. These steps were repeated for a total of 4 times.

The samples were treated as follows: fourteen mL of polymer solution were added to the top of the device. They were then centrifuged at 3500 RPM for four hours. The filtrate was recovered from the bottom of the device. The retentates were washed by adding 14 mL of 50 mM phosphate buffer, pH 7, and centrifuging for an additional 210 minutes. The filtrate was recovered from the bottom of the device. The retentates were recovered by inverting the top part of the device and centrifuging at 1000 RPM for 2 minutes. All fractions were analyzed by HPLC.

HPLC details:

A Dionex (Sunnyvale, Calif.) DX500 HPLC system equipped with a model PD-40 uv-visible photodiode array detector was used to monitor the synthetic efforts. This system was controlled with Dionex's Peaknet version 5.21 software. For the purposes of this work a Supelco TOSOH Biocep TSK-gel 7.8 mm×30 cm, 10 μM partical size column was utilized. The eluent was 50 mM phosphate buffer, 200 mM NaCl adjusted to pH=7.00 running at 0.6 ml/min with a run time of 35 minutes.

The conjugated polymers produced by the methods described herein can have a degree of conjugation of about 95% or higher. Such consistently high degrees of conjugation have not been acheived by prior art processes. The preferred highly conjugated Gd(DTPA)polylysine conjugates exhibit superior relaxivity in bulk water (6.8-7.8 Lmol⁻¹ sec⁻¹), as well as penetration in tumor tissues relative to comparable polymer of lower degrees of conjugation.

While specific embodiments of the invention have been illustrated and described herein, it is realized that modifications and changes will occur to those skilled in the art. It is therefore to be understood that the appended claims are intended to cover all such modifications and changes as fall within the true spirit and scope of the invention.

What we claim is:

1. A method comprising:
providing a substantially mono-activated steric hindrance molecule; and
reacting the substantially mono-activated steric hindrance molecule with a polymer,
to provide a polymer-steric hindrance molecule copolymer having an elongated structure and having a degree of conjugation of 90% or greater.

2. A method as in claim 1 wherein the steric hindrance molecule is a therapeutic agent.

3. A method as in claim 1 wherein the steric hindrance molecule is a targeting agent.

4. A method as in claim 1 wherein the steric hindrance molecule comprises an imaging agent.

5. A method as in claim 4 wherein the steric hindrance molecule chelates a paramagnetic entity.

6. A method as in claim 4 wherein the steric hindrance molecule chelates a lanthanide ion.

7. A method in claim 1 whrein the steric hindrance molecule is DTPA.

8. A method as in claim 1 wherein the polymer is a polypeptide.

9. A method as in claim 8 wherein the polypeptide is selected from the group consisting of polylysine, polyglutamic acid, polyaspartic acid and copolymers of lysine and either glutamic acid or aspartic acid.

10. A method comprising:
providing a substantially mono-activated lanthanide complexing molecule; and
reacting the substantially mono-activated lanthanide complexing molecule with a polypeptide.

11. The method of claim 10 wherein the step of providing a substantially mono-activated lanthanide ion complexing molecule comprises providing substantially mono-activated diethylenetriamine pentaacetic acid.

12. The method of claim 10 wherein the step of providing a substantially mono-activated lanthanide ion complexing molecule comprises providing an anhydride of diethylenetriamine pentaacetic acid.

13. The method of claim 12 wherein the step of providing an anhydride of diethylenetriamine pentaacetic acid activated with an amine comprises the steps of:
providing a solution containing diethylenetriamine pentaacetic acid and triethylamine;
cooling the solution to a temperature of about −40° C. or less; and
adding isobutyl chloroformate to the cooled solution.

14. The method of claim 10 wherein the step of reacting the substantially mono-activated lanthanide ion complexing molecule with a polypeptide comprises reacting the substantially mono-activated lanthanide ion complexing molecule with a polypeptide selected from the group consisting of polylysine, polyglutamic acid, polyaspartic acid and copolymers of lysine and either glutamic acid or aspartic acid.

15. The method of claim 10 wherein the step of reacting the substantially mono-activated lanthanide ion complexing molecule with a polypeptide comprises reacting the substantially mono-activated lanthanide ion complexing molecule with polylysine.

16. The method of claim 15 wherein the step of reacting the substantially mono-activated lanthanide ion complexing molecule with polylysine comprises adding the mono-activated lanthanide ion complexing molecule to an aqueous solution of polylysine.

17. The method of claim 10 further comprising the step of purifying the product produced by reacting the substantially mono-activated lanthanide ion complexing molecule with a polypeptide.

18. The method of claim 17 wherein the step of purifying the product produced by reacting the substantially mono-activated lanthanide ion complexing molecule with a polypeptide comprises removing impurities without substantially drying the product.

19. A lanthanide ion complexing molecule-polypeptide conjugate produced by the method of claim 10.

20. A method comprising:
providing a solution containing diethylenetriamine pentaacetic acid and triethylamine;
cooling the solution to a temperature of about −40° C. or less;
adding isobutyl chloroformate to the cooled solution to form a mono-anhydride of diethylenetriamine pentaacetic acid;
reacting the mono-anhydride of diethylenetriamine pentaacetic acid with a polypeptide containing lysine residues, whereby at least 90% of the lysine residues are substituted with diethylenetriamine pentaacetic acid groups.

21. The method of claim 20 wherein the step of reacting the substantially mono-activated diethylenetriamine pentaacetic acid with a polypeptide comprises reacting substantially mono-activated diethylenetriamine pentaacetic acid with polylysine.

22. The method of claim 20 further comprising the step of purifying the product produced by reacting the substantially mono-activated lanthanide ion complexing molecule with a polypeptide by removing impurities without substantially drying the product.

23. A diethylenetriamine pentaacetic acid-polypeptide conjugate produced by the method of claim 20.

24. A diethylenetriamine pentaacetic acid-ploysine conjugate having a degree of conjugation of about 95% or higher.

25. A method of imaging a subject comprising the steps of:
reacting a conjugated polymer produced by the method of claim 1 with a image producing entity; and
introducing the resulting product into a blood vessel of said subject.

26. A method as in claim 25 wherein the image producing entity is a paramagnetic entity.

27. A method as in claim 25 wherein the image producing entity is a lanthanide ion.

28. A method of concentrated delivery of an imaging agent to a tumor of a subject comprising the steps of:
reacting a diethylenetriamine pentaacetic acid-polypeptide conjugate produced by the method of claim 20 with a paramagnetic entity; and
introducing the resulting product into a blood vessel of said subject.

29. A method of purifying a conjugated polypeptide comprising the steps of:
subjecting a first composition to one or more purification processes without rendering the composition substantially dry, the first composition containing impurities and a polypeptide substituted with a steric hindrance molecule, the substituted polypeptide having an elongated configuration; and
recovering a purified composition containing the substituted polypeptide having an elongated configuration.

30. A method as in claim 29 wherein the step of subjecting the first composition to one or more purification processes without rendering the composition substantially dry comprises subjecting the first composition to one or more processes selected from the group consisting of evaporation, distillation, dialysis, centrifugation and ultrafiltration.

31. A method as in claim 29 wherein the first composition contains a polypeptide substituted with a steric hindrance molecule selected from the group consisting of diethylenetriaminepentaacetic acid (DTPA), 1,4,7,10-Tetraazacyclododecane-1,4,7,10-tetraacetic acid (DOTA), 1,4,7,10-Tetraazacyclododecane-1,4,7,10-tetrakis(2-propionic acid) (DOTMA), 1,4,8,11-tetraazacyclotetradecane-1,4,8,10-tetraacetic acid (TETA), 1,4,7,10-tetraazacyclododecane-1,4,7,10-tetrakis(3-(4-carboxyl)-butanoic acid), 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl-1,4,7,10-tetraazacyclododecane-1,4,7,10-tetraacetic acid (pSCN-Bz-DOTA), bis(thiosemicarbazone), derivatives of bis(thiosemicarbazone), porphyrins, derivatives of porphyrins, 2,3-bis(2-thioacetamido)propionates, derivatives of 2,3-bis(2-thioacetamido)propionates, N,N′-bis(mercaptoacetyl)-2,3-diaminopropanoate, bis(aminoethanethiol) and derivatives of bis(aminoethanethiol).
